(19) United States
(12) Patent Application Publication
(10) Pub. No.: US 2021/0040219 A1
(43) Pub. Date: Feb. 11, 2021

(54) IMPROVEMENTS IN CD47 BLOCKADE THERAPY BY EGFR ANTIBODY

(71) Applicant: Trillium Therapeutics Inc., Mississauga (CA)

(72) Inventors: Gloria Hoi Ying LIN, Oakville (CA); Natasja Nielsen VILLER, Etobicoke (CA); Mark Michael WONG, Toronto (CA); Robert Adam UGER, Richmond Hill (CA)

(21) Appl. No.: 16/979,754

(22) PCT Filed: Mar. 8, 2019
(86) PCT No.: PCT/CA2019/050287

§ 371 Date: Sep. 10, 2020

Related U.S. Application Data
(60) Provisional application No. 62/642,129, filed on Mar. 13, 2018.

(51) Int. Cl. C07K 16/28; A61K 38/17

(52) U.S. Cl. CPC C07K 16/2863; A61K 38/1774

(57) ABSTRACT

CD47+ disease cells such as cancer cells are treated using a combination of CD47 blocking agent and an EGFR antibody such as cetuximab. The anti-cancer effect of cetuximab is enhanced in the presence of SIRPαFc. Specific combinations include SIRPαFc forms that comprise an Fc that is either IgGl or preferably IgG4 isotype. These combinations are useful particularly to treat solid tumours and blood cancers including lymphomas, leukemias and myelomas.

### FIELD

This disclosure relates to methods and uses of a drug that blocks the CD47/SIRPα interaction. More particularly, the disclosure relates to methods and uses that, in combination, are useful for improving cancer therapy.

### BACKGROUND

Cancer cells are targeted for destruction by antibodies that bind to cancer cell antigens, and through recruitment and activation of macrophages by way of Fc receptor binding to the Fc portion of that antibody. Binding between CD47 on cancer cells and SIRPα on macrophages transmits a “don't eat me” signal that enables many tumour cells to escape destruction by macrophages. It has been shown that inhibition of the CD47/SIRPα interaction (CD47 blockade) will allow macrophages to “see” and destroy the target CD47+ cancer cell. The use of SIRPα to treat cancer by CD47 blockade is described in WO2010/130053.

Trillium Therapeutics' WO2014/094122 describes a protein drug that inhibits or antagonizes interaction between CD47 and SIRPα. This CD47 blocking agent is a form of human SIRPα that incorporates a particular region of its extracellular domain, linked with a particularly useful form of an IgG1-based Fc region. In this form, the SIRPαFc drug shows dramatic effects on the viability of cancer cells that present with a CD47+ phenotype. The effect is seen particularly on acute myelogenous leukemia (AML) cells, and many other types of cancer. A soluble form of SIRP having significantly altered primary structure and potent CD47 binding affinity is described in WO2013/19752.

Other CD47 blocking agents have been described, and these include various CD47 antibodies (see for instance Stanford's U.S. Pat. No. 8,562,997, and InhibRx′ WO2014/123580), each comprising different antigen binding sites but having, in common, the ability to compete with endogenous SIRPα for binding to CD47, to interact with macrophages and, ultimately, to increase CD47+ disease cell depletion. These CD47 antibodies have activities in vivo that are quite different from those intrinsic to drugs that incorporate SIRPα structure. The latter, for instance, display negligible binding to red blood cells whereas the opposite property in CD47 antibodies, and in high affinity SIRPα variants, creates a need for strategies that accommodate a drug “sink” that follows administration.

Still other agents are proposed for use in blocking the CD47/SIRPα axis. These include CD47Fc proteins described in Viral Logic's WO2010/083253, and SIRPα antibodies as described in University Health Network's WO2013/056352, Eberhard's U.S. Pat. No. 6,913,894, and elsewhere.

The CD47 blockade approach in anti-cancer drug development shows great clinical promise. There is a need to provide methods and means for improving the effect of these drugs, and in particular for exploiting the effect of the CD47 blocking agents that incorporate CD47-binding forms of SIRPα.

### SUMMARY

The anti-cancer effect of an anti-tumour antibody is improved when combined with a CD47 blocking agent. More particularly, the anti-cancer effect of an epidermal growth factor receptor (EGFR) antibody is improved when combined with a CD47 blocking agent in the form of SIRPαFc. This disclosure reveals that the anticancer effect of EGFR antibody is enhanced when administered in combination with a SIRPαFc. In embodiments, the SIRPαFc has an IgG4 isotype and comprises an IgV domain of human SIRPα, and the EGFR antibody is cetuximab. The enhancement of cetuximab activity caused by SIRPαFc manifests, for instance, as an increased depletion of treated EGFR+ cancer cells, a reduced rate of tumour growth, and/or as an enhanced survival in treated subjects, compared with results from either agent alone.

In one aspect, there is provided a method for treating a subject presenting with CD47+ disease cells, comprising administering to the subject a combination comprising an IgG4 isotype of SIRPαFc (designated SIRPαG4) and an EGFR antibody, such as cetuximab, including its marketed form, Erbitux®.

In a related aspect, there is provided the use of a SIRPαG4 in combination with an EGFR antibody for the treatment of a subject presenting with CD47+ disease cells such as cancer.

In another aspect there is provided a pharmaceutical combination comprising a SIRPαG4 and an EGFR antibody for use in the treatment of CD47+ disease cells.

There is also provided, in another aspect, a kit comprising a pharmaceutical combination comprising a SIRPαG4 and an EGFR antibody, together with instructions teaching their use in the treatment of CD47+ disease cells.

In a specific embodiment, the combination of the CD47 blocking agent and EGFR antibody is for use in the treatment of a solid tumour or a blood cancer such as a myeloma, a lymphoma or a leukemia.

In alternative embodiments, the SIRPαFc used in combination with an EGFR antibody is a SIRPαG1. In other alternative embodiments, the EGFR antibody is panitumumab, or its marketed form Vectibix®.

In related aspects, the present combination are used to treat cancer cells that are EGFR+, including cancer cells that are EGFR+ and CD47+.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF REFERENCE TO THE DRAWING

FIG. 1 shows that when Fadu (human head and neck tumor cell line) tumor bearing mice were treated with anti-EGFR antibody cetuximab (3 mg/kg, 2×/week for 2 weeks) in combination with SIRPαG4 (10 mg/kg, 5×/week for 6 weeks) starting on day 3 post tumor inoculation, increased tumor growth inhibition (A) and improved survival (B) were observed compared to anti-EGFR antibody cetuximab (3 mg/kg, 2x/week for 2 weeks) monotherapy or SIRPαG4 (10 mg/kg, 5×/week) monotherapy.

### DETAILED DESCRIPTION

The present disclosure provides methods, uses, combinations and kits useful for treating subjects that present with disease cells that have a CD47+ phenotype. In embodiments, the disease cells have a phenotype that is CD47+ and EGFR+. In this method, CD47+ cancer subjects receive a combination of an EGFR antibody such as cetuximab, and a CD47 blocking agent which preferably is an Fc-fused form of human SIRPα, i.e., SIRPαFc, in which the Fc is preferably an IgG4 isotype or an Fc receptor-binding variant thereof, designated SIRPαG4. The effect of the EGFR antibody is significantly enhanced by the CD47 binding SIRPαG4. This therapeutic effect is pronounced when the CD47+ disease cells are CD47+ cancer cells and tumours, and preferably EGFR+ cancer cells that are also CD47+ in phenotype.

The term “CD47+” is used with reference to the phenotype of cells targeted for binding by the present CD47 blocking agents. Cells that are CD47+ can be identified by flow cytometry using CD47 antibody as the affinity ligand. CD47 antibodies that are labeled appropriately are available commercially for this use (for example, the antibody product of clone B6H12 is available from BD Biosciences). The cells examined for CD47 phenotype can include standard tumour biopsy samples including particularly blood samples taken from the subject suspected of harbouring endogenous CD47+ cancer cells. CD47 disease cells of particular interest as targets for therapy with the present drug combination are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry or by immunostaining or by gene expression analysis or the like, to be greater than the level measurable on a counterpart cell having a CD47 phenotype that is normal for that cell type.

The term “CD47+ disease cells” thus refers to cells that are associated with a disease and have a CD47+ phenotype. In one embodiment, the CD47+ disease cells are cancer cells.

In embodiments, the CD47 blocking agent is an IgG4 version of human SIRPαFc, which interferes with and dampens or blocks signal transmission that would result when CD47 interacts with SIRPα. As described in Trillium Therapeutics' WO2014/094122, the entire contents of which are incorporated herein by reference, the preferred SIRPαG4 is an Fc fused form of a region of human SIRPα that interacts with CD47 and has been shown to have anti-cancer activity. The term “human SIRPα ” as used herein refers to a wild type, endogenous, mature form of human SIRPα. In humans, the SIRPα protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. The present disclosure is directed most particularly to the drug combinations that include the human SIRP variant 2 form, or V2.

In the present drug combination, the SIRPαFc fusion protein has a SIRPα component that comprises at least residues 32-137 of human SIRPα (a 106-mer), which constitute and define the IgV domain of the V2 form according to current nomenclature. This SIRPα sequence, shown below, is referenced herein as SEQ ID No.1.

[SEQ ID No. 1]
EELQVIQPDKSVSVAAGESAILHCTVTSLIPVGPIQWFRGAGPARELIYN
QKEGHFPRVTTVSESTKRENMDFSISISNITPADAGTYYCVKFRKGSPDT
EFKSGA

In a preferred embodiment, the SIRPαFc fusion protein incorporates the IgV domain as defined by SEQ ID No.1, and additional, flanking residues contiguous within the SIRPα sequence. This preferred form of the IgV domain, represented by residues 31-148 of the V2 form of human SIRPα, is a 118-mer having the sequence shown below:

[SEQ ID No. 2]
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPS.

The SIRPαFc protein incorporates an Fc region that has effector function. Fc refers to “fragment crystallisable” and represents the constant region of an antibody comprised principally of the heavy chain constant region and components within the hinge region. In embodiments, the Fc region includes the lower hinge-CH2-CH3 domains. More preferably, the Fc region includes the CH1-CH2-CH3 domains.

An Fc component “having effector function” is an Fc component having at least some natural or engineered function, such as at least some contribution to antibody-dependent cellular cytotoxicity or some ability to fix complement. Also, the Fc will at least bind to Fc receptors.

In embodiments, the Fc region comprises a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank. In one specific and preferred embodiment, the G4 Fc region incorporates an alteration at position 228 (EU numbering), in which the serine at this position is substituted by a proline (S228P), thereby to stabilize the disulfide linkage within the Fc dimer.

In a specific embodiment, the Fc region is based on the amino acid sequence of a human IgG4 set out as P01861 in UniProtKB/Swiss-Prot, residues 99-327, and has the amino acid sequence shown below and referenced herein as SEQ ID No.6:

[SEQ ID No. 3]
ESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

In an alternative embodiment, the SIRPαFc has an Fc region based on the amino acid sequence of a human IgG1 set out as P01857 in UniProtKB/Swiss-Prot, residues 104-330, and has the amino acid sequence shown below:

[SEQ ID No. 4]
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK*

In a specific embodiment, when the Fc component is an IgG4 Fc, the Fc incorporates at least the S²²⁸P mutation, and has the amino acid sequence set out below and referenced herein as::

[SEQ ID No. 5]
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

In a specific and preferred embodiment, the SIRPαFc fusion protein has the amino acid sequence set forth below: In this embodiment, the Fc component of the fusion protein is based on an IgG4, and incorporates the S²²⁸P mutation.

[SEQ ID No. 6]
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK

This SIRPαFc fusion protein is designated SIRPαG4.

In an alternative embodiment, the SIRPαFc fusion protein has the amino acid sequence set forth below: In this embodiment, the Fc component of the fusion protein is based on an IgG1:

[SEQ ID No. 7]
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK

This SIRPαFc fusion protein is designated SIRPαG1.

In a preferred embodiment, the SIRPαFc protein is provided and used in a secreted homodimeric fusion form, in which two copies of the fusion protein are coupled through covalent binding between cysteines present in separate SIRPαFc single polypeptide chains, e.g. SIRPαG4 chains having SEQ ID No.6.

The present drug combination comprises SIRPαG4, or SIRPαG1, as just described, and an agent such as and antibody that binds the human epidermal growth factor receptor (hEGFR), a protein that is presented on the surface of many different cell types including particularly skin cells such as keratinocytes. As used herein, the term “hEGFR” (EGFR+) refers to any protein that comprises the expressed and processed product of the human her-1 gene, wherein the protein is designated as UniProtKB/Swiss-Prot P00533. The term EGFR is used generically herein, and refers to the wild type protein and all naturally occurring variants thereof. The term “wtEGFR” is used more specifically with reference only to the wild type form of human EGFR. The term “EGFRvIII” refers to the EGFR variant protein that comprises the expressed and processed product of a variant of the her-1 gene lacking exons 2-7, and thus includes only the polypeptide sequence encoded by exons 1 and 8 of her-1. A disease cell that is EGFR+ is a disease cell that will bind cetuximab or any other antibody selective for EGFR binding.

For purposes of identifying disease cells that can be targeted by the present EGFR antibodies, the commercial test EGFRpharmDX (DAKO) can conveniently be used. This is a semi-quantitative immunohistochemical assay for determination of EGFR protein overexpression in colorectal tissues. Positive or negative results aid in the classification of abnormal cells/tissues and provide a basis for selecting tumours that are EGFR+.

The present combinations are based more particularly, and in one embodiment, on the hEGFR antibody known as cetuximab, now commercially available from Eli Lilly and Company under the trade name Erbitux®. Cetuximab is a recombinant, human/mouse chimeric IgG1 antibody that binds specifically to the extracellular domain of wtEGFR. The amino acid sequences the complete heavy chain (SEQ ID No.8) and complete light chain (SEQ ID No.9) of cetuximab.

[SEQ ID NO. 8]
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLEPPKPK
DTLMISRTPEVTCVVVDVSHEDPEVKFNWYVDGVEVENAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
[SEQ ID NO. 9]
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGE

In one embodiment, the SIRPαG4 is used in combination with either a formulated cetuximab, or the commercially formulated Erbitux®.

Erbitux® is sold as a sterile, clear, colorless liquid of pH 7.0 to 7.4, which may contain a small amount of easily visible, white, amorphous, cetuximab particulates. Each single-use, 50-mL vial contains 100 mg of cetuximab at a concentration of 2 mg/mL and is formulated in a preservative-free solution containing 8.48 mg/mL sodium chloride, 1.88 mg/mL sodium phosphate dibasic heptahydrate, 0.42 mg/mL sodium phosphate monobasic monohydrate, and Water for Injection, USP.

In an alternative embodiment, the SIRPαG4 is used in combination with the EGFR antibody known as panitumumab, now commercially available and sold under the trade name Vectibix®. Panitumumab is a recombinant, fully human IgG2 antibody that binds specifically to the extracellular domain of wtEGFR. The amino acid sequences of the heavy and light chains of panitumumab are listed in U.S. Pat. Nos. 6,235,883 and7,807,798, incorporated herein by reference.

Vectibix® is sold as a sterile, colorless, preservative-free solution containing 20 mg/mL Vectibix (panitumumab) in a single-use vial, e.g., a 5 mL single-use vial contains 100 mg of panitumumab in 5 mL (20 mg/mL); a 10 mL single-use vial contains 200 mg of panitumumab in 10 mL (20 mg/mL); and each 20 mL single-use vial contains 400 mg of panitumumab in 20 mL (20 mg/mL)

Each drug included in the present pharmaceutical combination can be formulated separately for use in combination. The drugs are said to be used “in combination” when the effect of each drug overlaps in a recipient of both drugs, and when the two drugs are combined in a physical mixture that is injectable or are provided in a separately packaged form such as in a kit. The combination can be produced by administering the EGFR antibody to the subject, followed by SIRPαFc administration, or vice versa.

The two drugs in the combination cooperate such that SIRPαG4 enhances the effect of cetuximab on target cells, and especially on target cancer cells that are CD47+ and EGFR+. This benefit manifests as a statistically significant improvement in a given parameter of target cell or tumour fitness or vitality. For instance, a benefit in CD47+/EGFR+ cancer cells, when exposed to a combination of CD47 blocking agent and EGFR antibody, can be a statistically significant decrease in the number of living cancer cells (hence a depletion), relative to non-treatment or single agent treatment, or a decrease in the number or size/volume of cancer cells or tumours, or an improvement in the endogenous location or distribution of any particular tumour type, or an enhancement in a survival parameter. In embodiments, the improvement resulting from treatment with the drug combination manifests as an effect that is at least additive and desirably synergistic, relative to results obtained when only SIRPαG4 or only cetuximab is used.

Particularly, SIRPαFc, such as SIRPαG4, can for example increase the efficacy of cetuximab on cetuximab resistant cancers or cells that develop resistance to cetuximab treatment. SIRPαG4 could also increase the efficacy of cetuximab on cells with lower levels of EGFR, by recruiting ADCC/ADCP mechanisms. SIRPαG4 may also increase the efficacy of cetuximab on cells that develop resistance to cetuximab via upregulation of alternative signaling pathways such as increased ERBB2 expression. Finally, it may increase efficacy of cetuximab in patients with EGFR having the KRAS mutation.

In use, each drug in the combination can be formulated as it would be for monotherapy, in terms of dosage size and form and regimen. In this regard, the improvement resulting from their combined use may permit the use of somewhat reduced dosage sizes or frequencies, as would be revealed in an appropriate clinical trial.

In this approach, each drug is provided in a dosage form comprising a pharmaceutically acceptable carrier, and in a therapeutically effective amount. As used herein, “pharmaceutically acceptable carrier” means any and all solvents, dispersion media, coatings, antibacterial and antifungal agents, isotonic and absorption delaying agents, and the like that are physiologically compatible and useful in the art of protein/antibody formulation. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. Each of the SIRPαG4 fusion protein and the EGFR antibody is formulated using practises standard in the art of therapeutics formulation. Solutions that are suitable for intravenous administration, such as by injection or infusion, are particularly useful.

Sterile solutions can be prepared by incorporating the active compound in the required amount in an appropriate solvent with one or a combination of ingredients noted above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation are vacuum drying and freeze-drying (lyophilization) that yield a powder of the active ingredient plus any additional desired ingredient from a previously sterile-filtered solution thereof.

As used herein, “effective amount” refers to an amount effective, at dosages and for a particular period of time necessary, to achieve the desired therapeutic result. A therapeutically effective amount of each drug in the combination may vary according to factors such as the disease state, age, sex, and weight of the individual, and the ability of the drug to elicit a desired response in the recipient. A therapeutically effective amount is also one in which any toxic or detrimental effects of the pharmacological agent are outweighed by the therapeutically beneficial effects. The EGFR antibody will of course be formulated in amounts that are suitable for patient dosing, as permitted by the regulatory agencies that have approved its use in humans. In use, each drug in the combination thus is formulated as it would be for monotherapy, in terms of dosage size and form and regimen. In this regard, the cooperation/benefit resulting from their combined use may permit the use of somewhat reduced dosage sizes or frequencies, as would be revealed in an appropriately controlled clinical trial.

The SIRPαFc fusion protein can be administered to the subject through any of the routes established for protein delivery, in particular intravenous, intradermal, intratumoural and subcutaneous injection or infusion, or by nasal administration.

Subjects targeted for treatment can be identified by first confirming the presence of disease cells that present with a phenotype that is at least CD47+ or EGFR+, and is ideally both CD47+ and EGFR+ sing the assays described above.

The drugs in the present combination can be administered sequentially or, essentially at the same time, i.e., concurrently or consecutively. In embodiments, the EGFR antibody is given before administration of the SIRPαFc. In the alternative, the EGFR antibody can be given after or during administration of the CD47 blocking agent, e.g., SIRPαG4. Thus, in some embodiments, the subject undergoing therapy is a subject already treated with one of the combination drugs, such as the EGFR antibody, and is then treated with the other of the combination drugs, such as the SIRPαFc drug. Most suitably, the drugs are administered such that their activities and actions overlap within the patient being treated, i.e., are in combination

Dosing regimens are adjusted to provide the optimum desired response (e.g., a therapeutic response). For example, a single bolus of each drug may be administered, or several divided doses may be administered over time or the dose may be proportionally reduced or increased as indicated by the therapeutic situation. It is especially advantageous to formulate parenteral compositions in unit dosage form for ease of administration and uniformity of dosage. “Unit dosage form” as used herein refers to physically discrete units suited as unitary dosages for the subjects to be treated; each unit contains a predetermined quantity of active compound calculated to produce the desired therapeutic effect in association with the required pharmaceutical carrier.

The drugs can be formulated in combination, e.g., as a kit, so that the combination can be introduced to the recipient in one administration, e.g., one injection or one infusion bag. Alternatively, the drugs can be combined as separate units in a kit that are provided together in a single package, and with written instructions teaching their use thereof according to the present method. In another embodiment, an article of manufacture containing the SIRPαFc drug and EGFR antibody combination in an amount useful for the treatment of the disorders described herein is provided. The article of manufacture comprises one or both drugs of the present antibody drug combination, as well as a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with the container indicates that the composition is used in combination with SIRPαFc drug in accordance with the present disclosure, thereby to elicit an enhanced effect on the CD47+ disease cells. The article of manufacture may further comprise a second container comprising a pharmaceutically-acceptable buffer, such as phosphate-buffered saline, Ringer's solution and dextrose solution. It may further include other matters desirable from a commercial and use standpoint, including other buffers, diluents, filters, needles, syringes, and package inserts with instructions for use.

For administration the dose for the SIRPαFc drug will be within the range from about 0.0001 to 100 mg/kg, and more usually 0.01 to 10 mg/kg, of the host body weight. For example, SIRPαFc dosages can be 0.3 mg/kg body weight, 1 mg/kg body weight, 3 mg/kg body weight, 5 mg/kg body weight or 10 mg/kg body weight or within the range of 0.1-100 mg/kg. When the CD47 blocking agent is a SIRPαFc fusion protein of SEQ ID No.6, the dose can be about 1 ug-5 mg per injection, such as intratumoural injection.

The SIRPαFc protein displays negligible binding to red blood cells. There is accordingly no need to account for an RBC “sink” when dosing with the drug combination. Moreover, the SIRPα -Fc fusion protein is a dedicated antagonist of the SIRPα -mediated signal, as it displays negligible CD47 agonism when binding thereto. There is accordingly no need, when establishing medically useful unit dosing regimens, to account for any stimulation induced by the drug.

Administration of cetuximab to a subject will typically entail a loading dose of 400 mg/m2 (2 hr IV infusion) and a maintenance dose of 250 mg/m2 (1 hr IV infusion) every week.

The drug combination is useful to treat a variety of CD47+ disease cells including CD47+ disease cells that are also EGFR+, and disease cells that are EGFR+ only. These include particularly CD47+ cancer cells, including liquid and solid tumours. Solid tumours can be treated with the present drug combination, to reduce the size, number, distribution or growth rate thereof and to control growth of cancer stem cells. Such solid tumours include CD47+ tumours such as carcinomas in skin (melanoma), bladder, brain, breast, lung, colon, ovary, prostate, head and neck, colorectal tissue, liver and other tissues as well. In one embodiment, the drug combination can used to inhibit the growth or proliferation of hematological cancers. As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome. “Lymphoma” may refer to a Hodgkin's lymphoma, both indolent and aggressive non-Hodgkin's lymphoma, cutaneous T cell lymphoma (CTCL), peripheral T cell lymphoma (PTCL) Burkitt's lymphoma, Mantle cell lymphoma (MCL) and follicular lymphoma (small cell and large cell), among others including DLBCL and FL. Myelomas include multiple myeloma (MM), giant cell myeloma, heavy-chain myeloma, and light chain myeloma and Bence-Jones myeloma.

In some embodiments, the hematological cancer treated with the drug combination is a CD47+ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with the drug combination is a CD47+ lymphoma or myeloma selected from Hodgkin's lymphoma, both indolent and aggressive non-Hodgkin's lymphoma, diffuse large cell lymphoma (DLBCL), mantle cell lymphoma, T cell lymphoma including mycosis fungoides, Sezary's syndrome, Burkitt's lymphoma, follicular lymphoma (small cell and large cell), multiple myeloma (MM), giant cell myeloma, heavy-chain myeloma, and light chain or Bence-Jones myeloma as well as leimyosarcoma. When the cancer is a carcinoma, the disease can include Merkel cell carcinoma, squamous cell carcinoma, and soft tissue carcinoma.

In a specific embodiment, the cancer treated with the present combination is multiple myeloma. In another specific embodiment, the targeted cancer is mantle cell lymphoma. In a further embodiment, the cancer treated with the present combination is relapsed or refractory Hodgkin's lymphoma. In another specific embodiment, the CD47 blocking agent is SIRPαFc. In a further specific embodiment, the EGFR antibody is cetuximab.

In some embodiments, the treated cancer is an EGFR+ cancer, such as colorectal, head and neck, lung, breast and glioma, and any other cancer for which treatment with an EGFR antibody is indicated.

In still other embodiments, cetuximab is used in combination with SIRPαFc, such as SEQ ID No.6 or SEQ ID No.7, such as for the treatment of cutaneous T cell lymphoma or multiple myeloma. In another embodiment, the combination is used to treat a T cell lymphoma such as mycosis fungoides or Sezary's syndrome.

Thus, in specific embodiments, there is provided the use of a CD47 blocking agent in combination with an EGFR antibody for the treatment of a particular CD47+ cancer, wherein:
i) the CD47 blocking agent is SIRPαG4 of SEQ ID No.6 and the EGFR antibody is cetuximab, such as for the treatment of a cancer that is head and neck cancer, colorectal cancer, cutaneous T cell lymphoma or multiple myeloma or relapsed or refractory Hodgkin's lymphoma.;
ii) the CD47 blocking agent is SIRPαG1 of SEQ ID No.7 and the EGFR antibody is cetuximab, such as for the treatment of a cancer that is head and neck cancer, colorectal cancer, cutaneous T cell lymphoma or multiple myeloma or relapsed or refractory Hodgkin's lymphoma.;
iii) the CD47 blocking agent is SIRPαG4 of SEQ ID No.6 and the EGFR antibody is panitumumab, such as for the treatment of a cancer that is head and neck cancer, colorectal cancer, cutaneous T cell lymphoma or multiple myeloma.

It will be appreciated that other SIRPαFc-based blocking agents can be used in combination with an EGFR antibody. Desirable combinations will show a statistically significant improvement in cancer cell response. This can be demonstrated as a statistically significant improvement in EGFR antibody activity caused by combination with a CD47 blocking agent, or vice versa, where statistical significance is shown as noted in the examples that follow and desirably, provides a p value >0.05 and more desirably >0.01 such as >0.001.

The combination therapy, comprising CD47 blockade and EGFR inhibition can also be exploited together with any other agent or modality useful in the treatment of the targeted indication, such as surgery as in adjuvant therapy, or with additional chemotherapy as in neoadjuvant therapy.

The following non-limiting example illustrates the present disclosure.

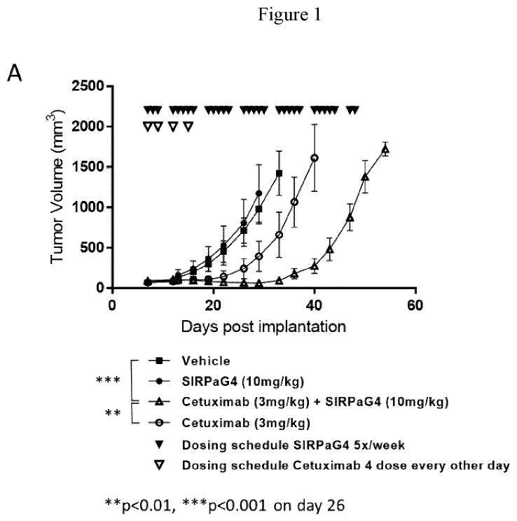

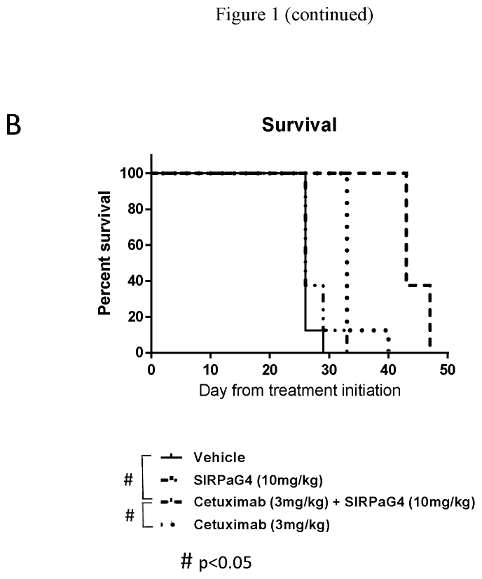

With reference to FIG. 1, 5×10⁶ Fadu cells in Matrigel were implanted subcutaneously into the right flank of NOD SCID (n=8 mice per group) on day 0. Mice were randomized on day 3 and received intraperitoneal (IP) injections of SIRPαG4 10 mg/kg 5x/week or/and Cetuximab 3 mg/kg 4 doses every other day) or vehicle 5×/week. (FIG. 1A) The mean tumor volume with standard deviation of each treatment group is shown: SIRPαG4 10 mg/kg 5×/week (black circles) or/and cetuximab (3 mg/kg 4 doses every other day) or vehicle 5×/week (gray squares). The curve terminates when >25% of animals per group were sacrificed. Statistical significance was calculated by one-way ANOVA (Tukey's multiple comparisons test) based on tumor volumes on day 26. (FIG. 1B) Enhanced survival of the tumor bearing mice of each treatment group is also shown: SIRPαG4 10 mg/kg 5×/week (dash with dotted line), Cetuximab 3 mg/kg 4 doses every other day (dotted line), SIRPαG4 10 mg/kg 5×/week and Cetuximab 3 mg/kg 4 doses every other day (dashed line) or vehicle 5×/week (solid line). Statistical significance of the survival curves was calculated by LogRank test using Prism GraphPad software.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method for treating a subject presenting with disease cells that are CD47+ and EGFR+, comprising administering to the subject a combination of a SIRPαFc protein and an epidermal growth factor receptor (EGFR) antibody.

2. The method according to claim 1, wherein the EGFR antibody is cetuximab.

3. The method according to claim 1, wherein the EGFR antibody is Erbitux®.

4. The method according to claim 1, wherein the EGFR antibody is panitumumab.

5. The method according to claim 1, wherein the EGFR antibody is Vectibix®.

6. The method according to claim 1, wherein the SIRPαFc drug comprises SEQ ID No. 6.

7. The method according to claim 1, wherein the SIRPαFc drug comprises SEQ ID No.7.

8. The method according to claim 1, wherein the CD47+ disease cells are cancer cells.

9. The method according to claim 8, wherein the cancer cells are blood cancer cells or solid tumour cells, optionally head and neck cancer cells or colorectal cancer cells.

10. The method according to claim 9, wherein the cancer cells are blood cancer cells.

11. The method according to claim 10, wherein the blood cancer cell is a leukemia, a lymphoma or a myeloma.

12. The method according to claim 11, wherein the leukemia is selected from acute lymphocytic leukemia (ALL); acute myeloid leukemia (AML); chronic lymphocytic leukemia (CLL); and chronic myelogenous leukemia (CML).

13. The method according to claim 11, wherein the cancer is a lymphoma selected from a Hodgkin's lymphoma, both indolent and aggressive non-Hodgkin's lymphoma, Burkitt's lymphoma, and follicular lymphoma (small cell and large cell).

14. The method according to claim 11, wherein the cancer is a myeloma selected from multiple myeloma (MM), giant cell myeloma, heavy-chain myeloma, light chain or Bence-Jones myeloma, myeloproliferative disorder/neoplasm (MPDS); and myelodysplastic syndrome.

15. The method according to claim 1, wherein the EGFR antibody is administered to a subject that has already received the SIRPαFc drug.

16. (canceled)

17. A pharmaceutical combination comprising an effective amount of a SIRPαFc drug, and an effective amount of an EGFR antibody.

18. The combination according to claim 17, wherein the EGFR antibody is cetuximab.

19. The combination according to claim 17, wherein the SIRPαFc drug comprises SEQ ID No. 6.

20. The combination according to claim 17, wherein the SIRPαFc drug comprises SEQ ID No. 7.

21. A kit comprising a combination of claim 17, and written instructions for the use thereof for the treatment of a subject presenting with CD47+ disease cells.

22. (canceled)


SEQUENCE LISTING (9 sequences)

SEQ ID NO: 1 - protein, 106 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 3 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 347 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 345 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, Homo sapiens
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE